(19) United States
(12) Patent Application Publication
(10) Pub. No.: US 2016/0354228 A1
(43) Pub. Date: Dec. 8, 2016

(54) DEVICE FOR HEALING OF DECUBITUS AND OTHER NON-HEALING ULCERS AND METHOD OF USE THEREIN

(71) Applicant: Abbas M. HUSAIN, Voorhees, NJ (US)

(72) Inventor: Abbas M. HUSAIN, Voorhees, NJ (US)

(21) Appl. No.: 15/173,254

(22) Filed: Jun. 3, 2016

Related U.S. Application Data
(60) Provisional application No. 62/170,487, filed on Jun. 3, 2015.

(51) Int. Cl. A61F 5/34; A61M 39/22

(52) U.S. Cl. CPC A61F 5/34; A61M 39/22

(57) ABSTRACT

A device for healing of decubitus and other non-healing ulcers includes a protective wrap that has air filled bubbles that cushion and relive pressure on a patient that is wrapped therein. In one embodiment, a full body wrap covers almost all of the patient. A torso only embodiment allows the patient's legs to remain free while providing healing for any ulcer located on the torso. In use, the patient is easily rolled into different positions to relieve pressure by simply rotating them since they are enclosed in a cylindrical wrap. The patient lies on the open protective wrap and then a left and right portion is wrapped around the patient and secured using a hook and loop fastener. The protective wrap is used and disposed of. The wrap may be cleaned and used again, but should not be used with multiple patients.

### CROSS REFERENCE TO RELATED APPLICATIONS

This application claims priority and herein incorporates by reference U.S. provisional patent application 62170487, filed Jun. 3, 2015.

### BACKGROUND OF THE INVENTION

Decubitus ulcers, also known as pressure ulcers or bed sores, are a very prevalent problem in nursing homes. All long-term patients especially paralyzed patients are prone to these ulcers because they are lying in the same position, motionless for hours and days at a time. Other non-healing ulcers such as bed sores, etc. also are very difficult to treat and can be very painful and of concern to patients. There is a need for an effective easy to use device that promotes healing of non-healing ulcers that are very hard to treat.

### SUMMARY OF THE INVENTION

A device for healing of decubitus and other non-healing ulcers includes a protective wrap that has air filled bubbles that cushion and relive pressure on a patient that is wrapped therein. In one embodiment, a full body wrap covers almost all of the patient. A torso only embodiment allows the patient's legs to remain free while providing healing for any ulcer located on the torso. In use, the patient is easily rolled into different positions to relieve pressure by simply rotating them since they are enclosed in a cylindrical wrap. The patient lies on the open protective wrap and then a left and right portion is wrapped around the patient and secured using a hook and loop fastener. The protective wrap is used and disposed of. The wrap may be cleaned and used again, but should not be used with multiple patients.

Other features and advantages of the instant invention will become apparent from the following description of the invention which refers to the accompanying drawings.

### BRIEF DESCRIPTION OF THE DRAWINGS

FIG. 1 is a transparent front side view of a patient enclosed in a full body device for healing of decubitus and other non-healing ulcers according to an embodiment of the invention.

FIG. 2 is a transparent front side view of a patient enclosed in a torso device for healing of decubitus and other non-healing ulcers according to an embodiment of the invention.

FIG. 3 is a top view of the device for healing decubitus and other non-healing ulcers shown in FIG. 1.

FIG. 4 is a side view of the device for healing decubitus and other non-healing ulcers shown in FIG. 1 before surrounding a patient.

FIG. 5 is a bottom view of the device for healing decubitus and other non-healing ulcers shown in FIG. 4.

FIG. 6 is a bottom view of the device for healing decubitus and other non-healing ulcers shown in FIG. 5 after rolling the right side.

FIG. 7 is a bottom view of the device for healing decubitus and other non-healing ulcers shown in FIG. 5 after rolling the right and left side and surrounding the patient.

FIG. 8 bottom view of the device for healing decubitus and other non-healing ulcers shown in FIG. 5 secured with straps.

FIG. 9 is a front side view of the device for healing decubitus and other non-healing ulcers shown in FIG. 2 before surrounding a patient.

FIG. 10 is a top view of a patient enclosed in a full body device for healing of decubitus and other non-healing ulcers with multiple cushioning layers according to an embodiment of the invention.

FIG. 11 is a top view of the device for healing of decubitus and other non-healing ulcers shown in FIG. 1 having a filling valve.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

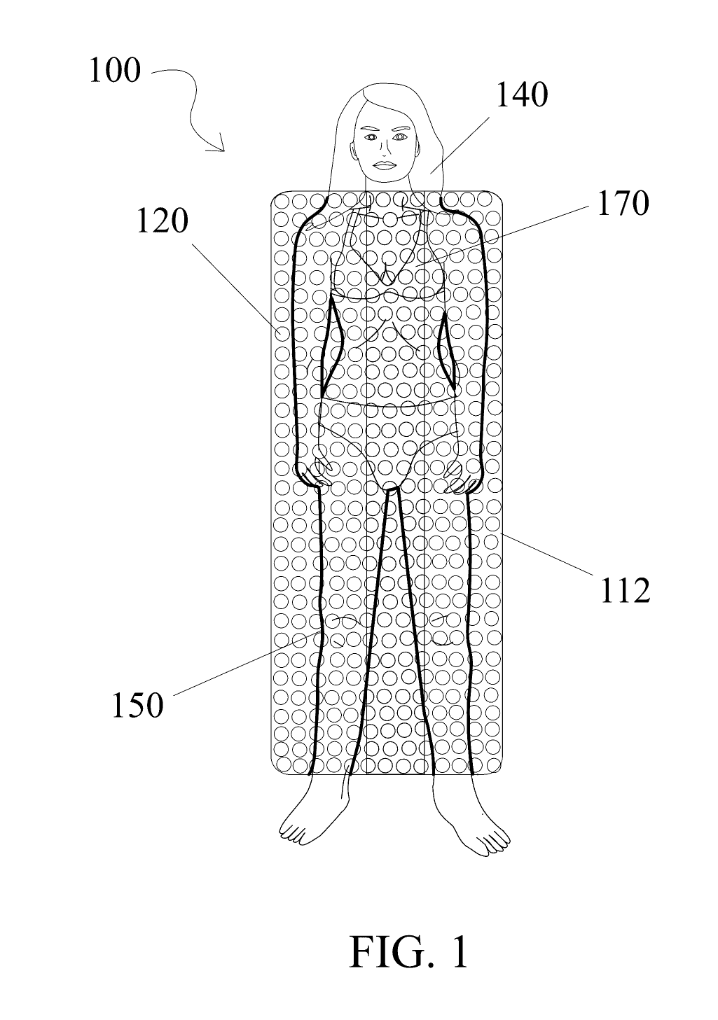

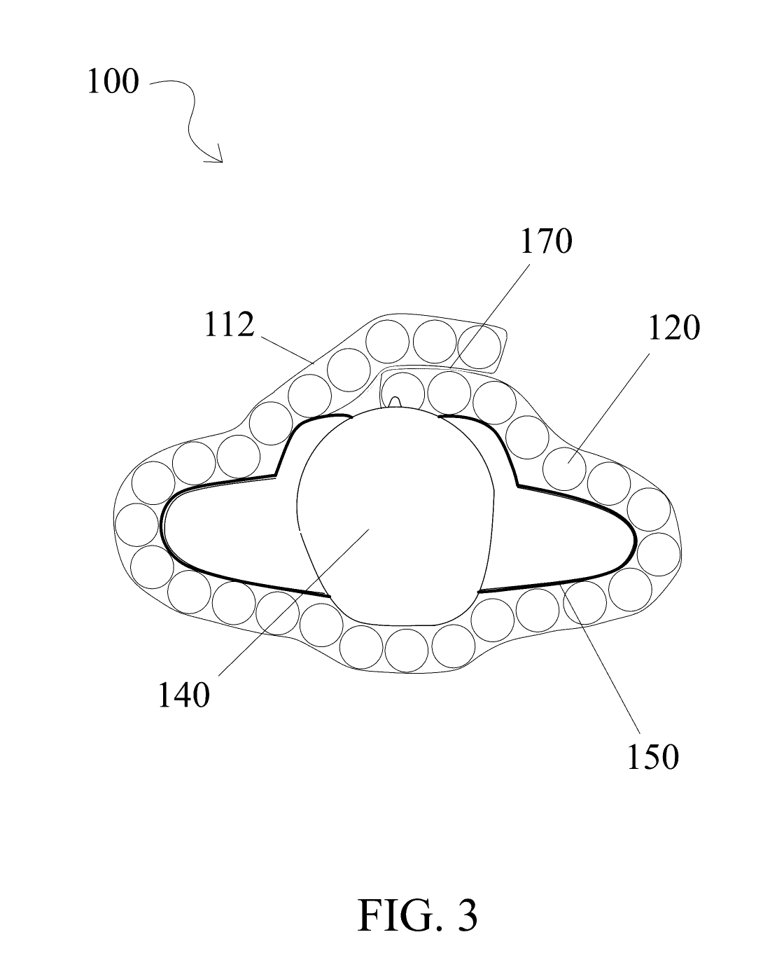

Referring to FIGS. 1 and 3, a device for healing of decubitus and other non-healing ulcers 100 is shown having a full body protective wrap 112 that contains compression bubbles 120 that are filled with a gas such as air. Compression bubbles 120 may alternatively be filled with a gel. In the gel embodiment, temperature regulation may be enhanced by the cooling properties of the gel. A patient 140 is wrapped up in protective wrap 112 with an inner portion of protective wrap 112 that forms a skin contact area 150. Protective wrap 112 is secured with a hook and loop portion 170 that is disposed on the edges of protective wrap 112. Although hook and loop portion 170 is shown along a complete left and right side, it is understood that it is not necessary to cover the entire portion and may cover only a selected area or multiple areas to secure patient therein.

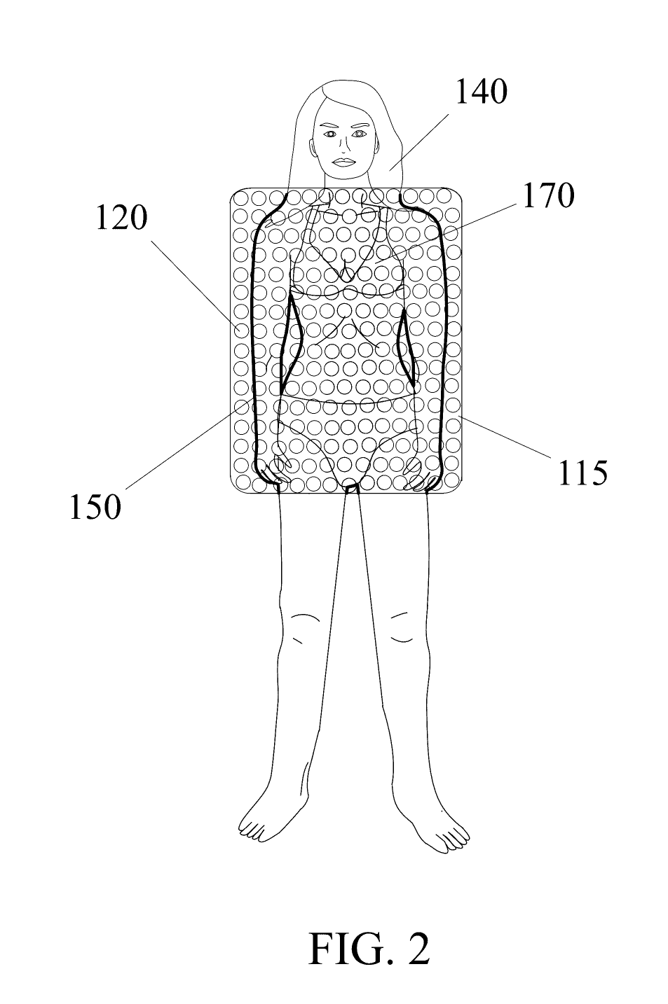

Now referring to FIG. 2, a device for healing of decubitus and other non-healing ulcers is basically the same as the device shown in FIG. 1, except that protective wrap 115 only covers the patient's torso rather than the full body. Torso protective wrap 115 also contains compression bubbles 120 that are air filled as discussed above. Patient 140 is wrapped up in torso protective wrap 115 with an inner portion of protective wrap 115 that forms skin contact area 150. Torso protective wrap 115 is secured with hook and loop portion 170 that is disposed on the edges of protective wrap 115

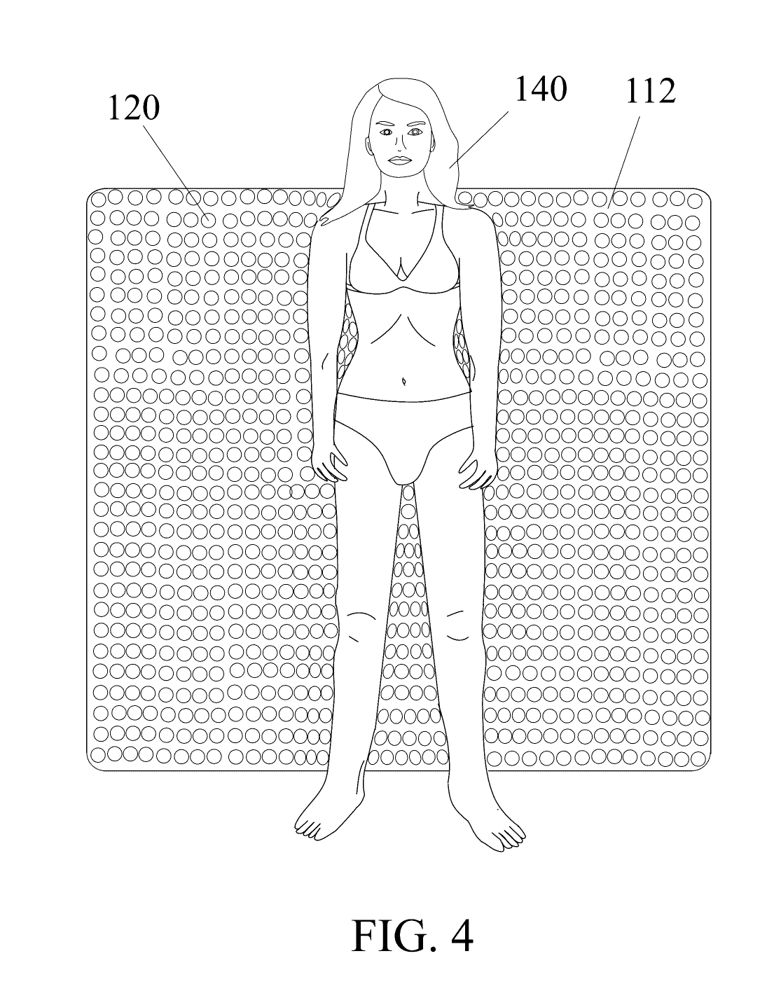

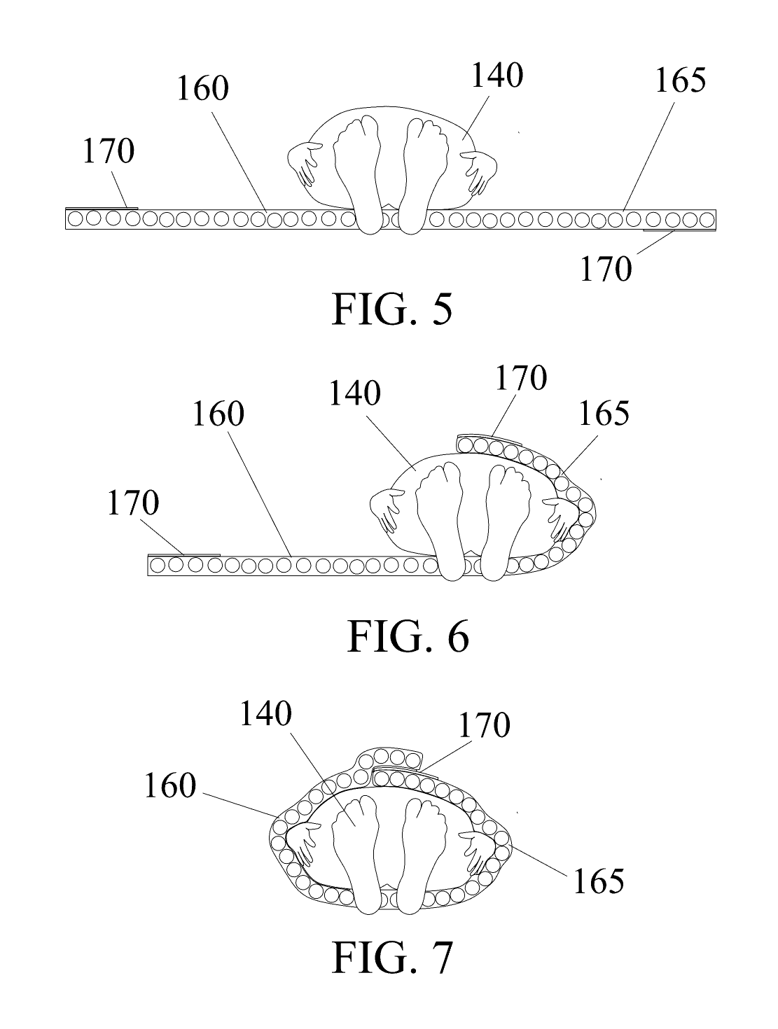

Referring now to FIGS. 4 through 7, patient 140 is laid on protective wrap 112 that includes compression bubbles 120 that provide cushioning and protection. A protective wrap left side 160 and protective wrap right side 165 allows patient 140 to be enclosed within. As is shown in the figures, protective wrap right side 165 is wrapped around patient and then protective wrap left side 160 is wrapped and secured with hook and loop portion 170 to provide an fully enclosed protective wrap that allows patient 140 to be easily rotated to relieve pressure and promote healing.

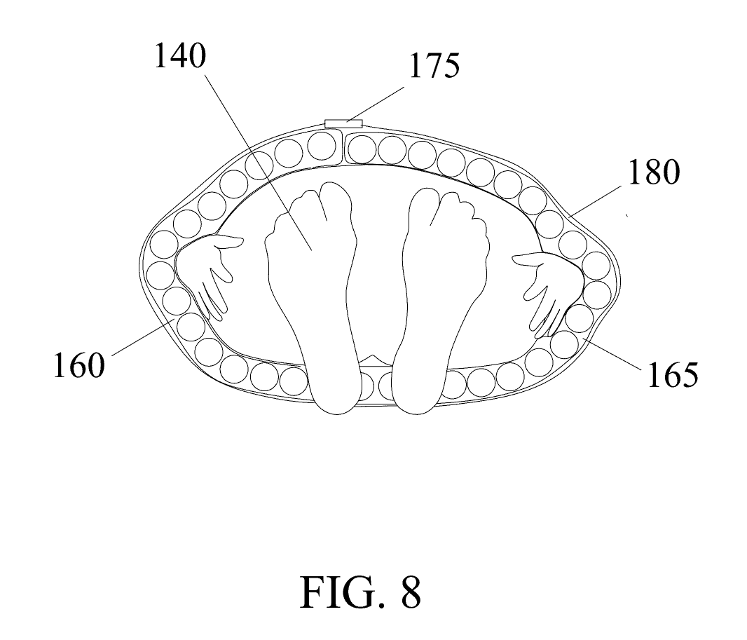

Referring to FIG. 8, a strap 175 is used to secure protective wrap left side 160 and protective wrap right side 165 to patient 140 instead of hook and loop fastener 170. Of course other securing methods may be used to hold protective wrap together such as, but not limited to snaps, ties, laces, etc. as is known in the art.

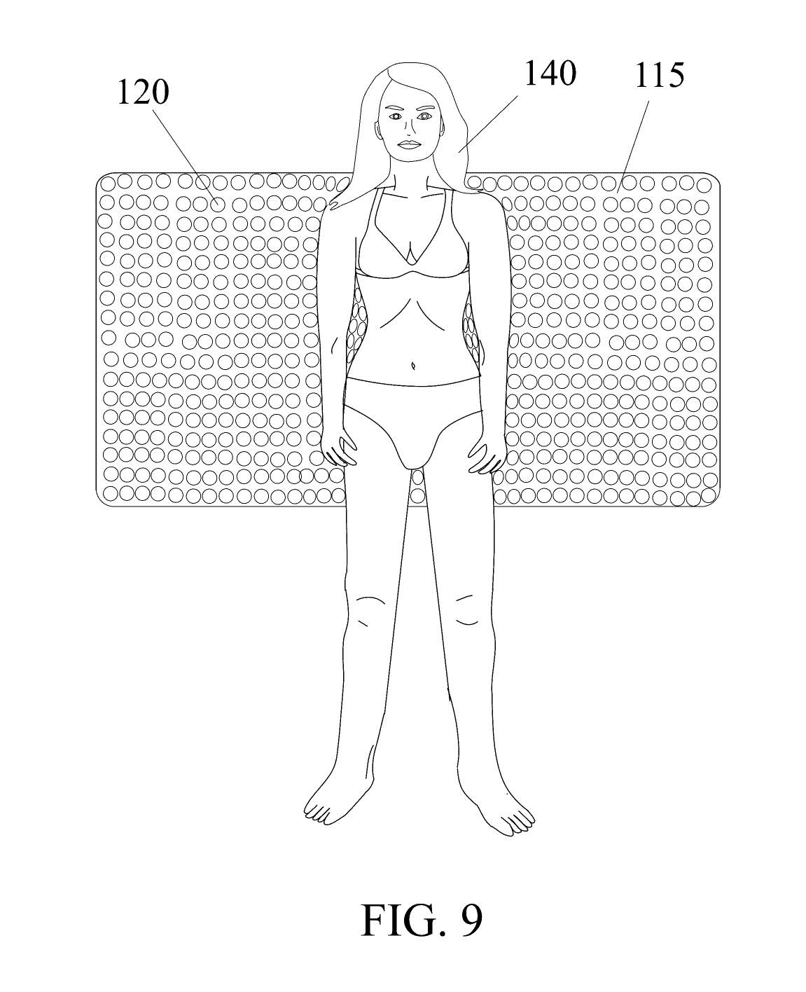

Now referring to FIG. 9, patient 140 is shown being placed on torso protective wrap 115 as discussed above. This embodiment allows patient to maintain leg movement when they are not involved in decubitus or other non-healing ulcer.

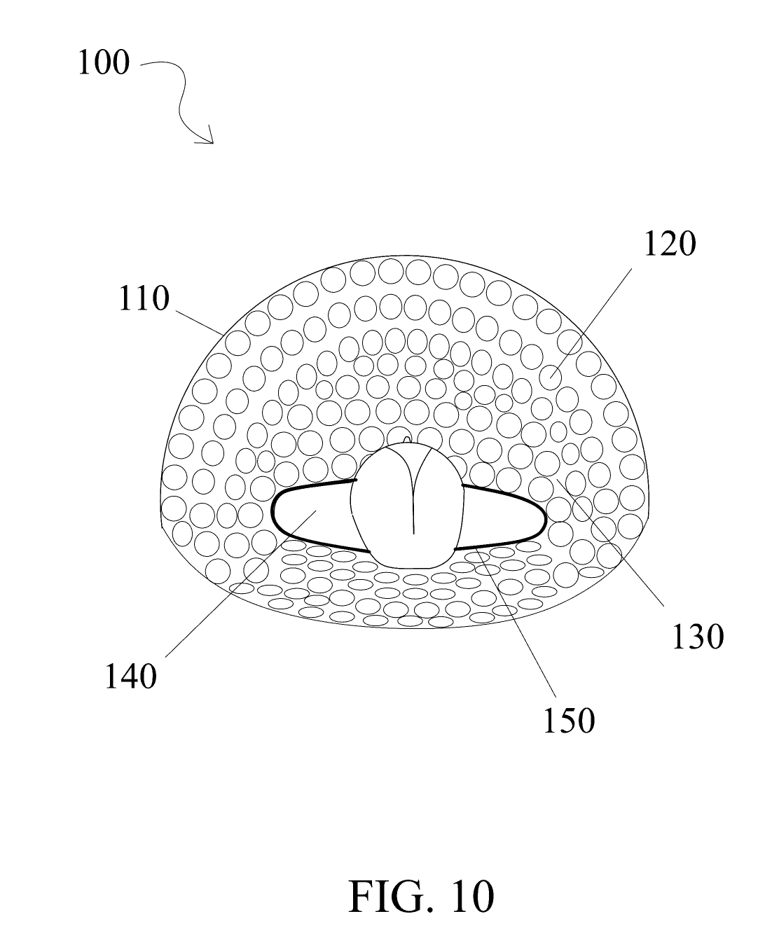

FIG. 10 illustrates an embodiment of a device for healing of decubitus and other non-healing ulcers where patient 140 is wrapped with multiple layers of compression bubbles to provide more cushioning and allows patient 140 to rotate more easily since a more cylindrical shape is achieved. This embodiment is representative to multiple layers and may be simply more than one layer and not necessarily as many as are shown in the figure.

Device for healing of decubitus and other non-healing ulcers is generally disposable and may be used once and disposed of to promote sterility in a hospital or other care environment. It may also be desirable to reuse protective wrap with the same patient as long as it is carefully cleaned between wrappings. It is important to not re-use it with multiple patients since the risk of infection increases with ulcers.

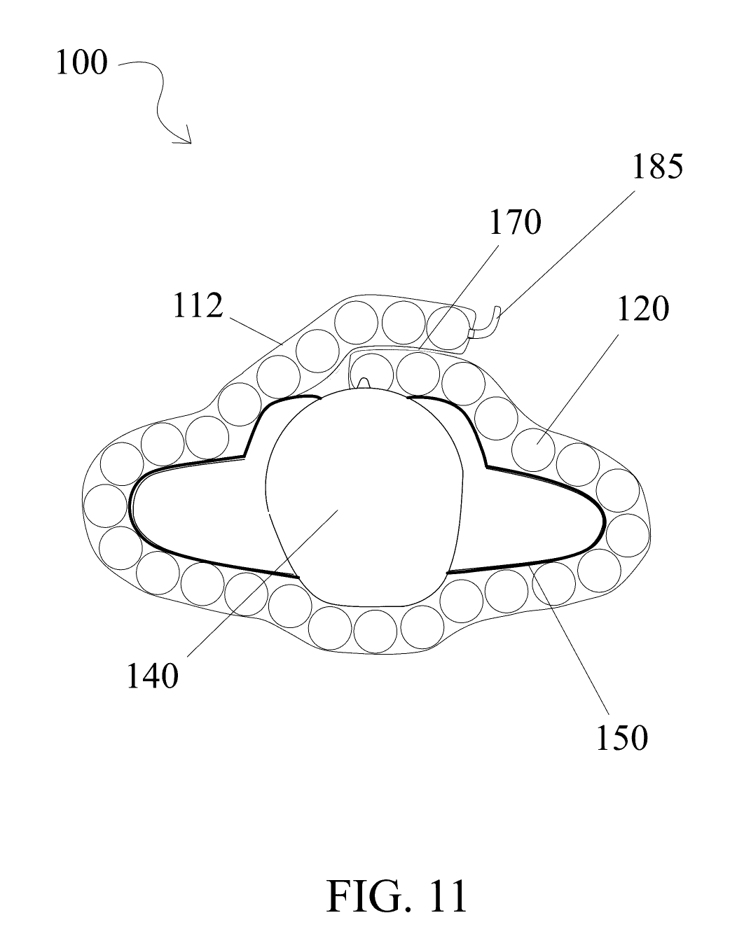

Referring to FIG. 11, the compression bubbles may be factory filled or may be inflated using an inflation valve 185 just prior to use to enhance storage and transport as is known in the art. Alternatively, the patient may be placed on wrap and then inflated to fully support and fit against the patient's skin. Additionally, the shape of the compression bubbles is not critical and although shown as circular, they may be any shape including, but not limited to, ovals, cylinders, elliptical or any other suitable shape.

The protective wrap is made of medical grade polyurethane, nitrile, polyisoprene or other suitable material that will encourage and promote healing when placed in contact with the patient's skin. In use, once the patient is wrapped within, the patient may be easily rotated to relieve pressure. Because the patient is wrapped and forms a cylindrical shape, even one patient care provider can rotate the patient on a regular schedule to promote healing of ulcers that are generally very hard to deal with. Traditional methods of rearranging a patient can be difficult and cause additional stress to the patient.

Although the instant invention has been described in relation to particular embodiments thereof, many other variations and modifications and other uses will become apparent to those skilled in the art.

What is claimed is:

1. A device for healing of decubitus and other non-healing ulcers comprising:
a protective wrap:
said protective wrap having a selected dimension;
said protective wrap having a left side and a right side;
said protective wrap having a plurality of compression bubbles disposed therein; and
a securing means for securing said patient within said protective wrap.

2. The device for healing of decubitus and other non-healing ulcers according to claim 1 wherein said compression bubbles are filled with air.

3. The device for healing of decubitus and other non-healing ulcers according to claim 1 wherein said compression bubbles are filled with gel.

4. The device for healing of decubitus and other non-healing ulcers according to claim 1 wherein said securing means is a hook and loop fastener.

5. The device for healing of decubitus and other non-healing ulcers according to claim 1 wherein said selected dimension is selected to fit over said patient's full body.

6. The device for healing of decubitus and other non-healing ulcers according to claim 1 wherein said selected dimension is selected to fit over said patient's torso only.

7. The device for healing of decubitus and other non-healing ulcers according to claim 1 wherein said securing means further comprises at least one strap.

8. The device for healing of decubitus and other non-healing ulcers according to claim 1 wherein said compression bubbles are filled with a gel.

9. The device for healing of decubitus and other non-healing ulcers according to claim 1 wherein said protective wrap is made of polyurethane.

10. The device for healing of decubitus and other non-healing ulcers according to claim 1 wherein said protective wrap is made of nitrile.

11. The device for healing of decubitus and other non-healing ulcers according to claim 1 wherein said protective wrap is made of polyisoprene.

12. The device for healing of decubitus and other non-healing ulcers according to claim 1 further comprising an inflating valve disposed on said protective wrap and in fluid contact with said plurality of compression bubbles.

13. A method of promoting healing of decubitus and other non-healing ulcers comprising the steps of:
obtaining a protective wrap;
said protective wrap having a selected dimension;
said protective wrap having a left side and a right side;
said protective wrap having a plurality of compression bubbles disposed therein; and
a securing means for securing said patient within said protective wrap;
opening said protective wrap to provide a flat surface;
placing said patient centrally on said protective wrap;
wrapping said left and said right side around said patient;
securing said patient therein using said securing means; and
rotating said patient to a new position to relieve pressure on portions of said patient.

14. The method of healing of decubitus and other non-healing ulcers according to claim 13 further comprising the step of inflating said plurality of compression bubbles where an inner portion of said protective wrap is adapted to fit tightly against said patient's skin.

15. The method of healing of decubitus and other non-healing ulcers according to claim 13 further comprising the steps of disposing of said protective wrap after use and obtaining a new protective wrap.
